(19) United States
(12) Patent Application Publication
(10) Pub. No.: US 2006/0074029 A1
(43) Pub. Date: Apr. 6, 2006

(54) Topical antimicrobial composition having improved moisturization properties

(76) Inventor: Scott Leece, Chicago, IL (US)

Correspondence Address: Kimberly Luna, Paralegal;Allegiance Corporation, 1430 Waukegan Road, McGaw Park, IL, 60085, US

(21) Appl. No.: 10/957,526

(22) Filed: Oct. 4, 2004

(51) Int. Cl. A61K 31/70; A61K 31/045

(52) U.S. Cl. 514/23; 514/724

(57) ABSTRACT

The invention described herein provides an extended resident topical antimicrobial composition comprising an active antimicrobial and moisturizing ingredients that meets antimicrobial efficacy requirements and improves the condition of the user's skin as well. The composition of the invention achieves desired antimicrobial efficacy and therapeutic effect and still exhibits a relatively short dry time to avoid unpleasant feel. The composition of the invention comprises: a) an antimicrobial agent; and b) a moisturization component comprising a combination of a saccharide isomerate and carbohydrate complex together with glycerin. In a preferred embodiment, the antimicrobial agent is a broad spectrum antimicrobial agent such as alcohol. The invention is particularly useful to healthcare workers, whose usage of hand sanitizers can be on a daily and frequent basis.

### FIELD OF THE INVENTION

The invention herein relates to the field of topical antimicrobial compositions applied to the user's skin. In particular, the invention pertains to a topical antimicrobial composition that, in addition to exhibiting desirable levels of antimicrobial efficacy, exhibits improved moisturization and feel.

### BACKGROUND OF THE INVENTION

In certain fields, such as the medical and food preparation fields, it is desirable to lower the risks of microbial contact during the performance of various tasks. A variety of techniques or methods to maintain cleanliness are well known, for example, washing hands, donning gloves, and the like. Various topical antimicrobial compositions and antimicrobially effective ingredients are well known in the art. Some compositions are typically formulated as soaps or products that are intended to have short term residence on the skin before they are rinsed off.

Another method of reducing the presence of microbes on the skin, e.g., bacteria, viruses, fungus, etc., is through the use of topical antimicrobial compositions intended to remain on the user's skin. These products reduce the amount of infectious organisms on healthcare worker's hands, for example, thus reducing the likelihood of healthcare-acquired infection. Commonly referred to as “hand sanitizers”, such compositions are often used by healthcare workers to clean their hands throughout the day when washing with soap and water is not convenient. Examples of such hand sanitizer products include Purell® (available from Gojo Industries, Inc., Akron, Ohio) and Endure® 320 (available from Ecolab, Inc., St. Paul, Minn.).

Because such compositions are applied onto the skin surface and remain thereafter, the active antimicrobial ingredient(s) also come into extended contact with the skin surface. Certain ingredients, such as ethanol, chlorhexidine gluconate, and the like, have been known to produce irritation and moisture loss on the user's skin.

Because an applied composition in the form of a lotion, for example, has an extended topical residence, undesirable effects such as loss of skin moisture is even more likely. For those individuals that repeatedly or frequently use such compositions, whether out of necessity or hygiene preference, the impact on the condition of the user's skin can be more aggravated. Alcohol-based hand sanitizers are particularly problematic, since alcohol is a drying agent. It is known that frequent and repeated use of alcohol-based hand sanitizers can result in skin disorders, such as contact dermatitis.

Another problem associated with existing topical antimicrobial compositions is the balance of attaining a desirable threshold of broad spectrum antimicrobial efficacy (kills gram positive bacteria, gram negative bacteria, yeast) while reducing the adverse effects of the composition ingredients on the skin. Another problem associated with certain compositions is that while moisturizing agents can be added or incorporated alongside the active antimicrobial agent, the moisturizers can also interfere with the antimicrobial effect. Furthermore, many moisturizing agents can produce a greasy, sticky, or otherwise unpleasant sensation on the skin following their application to the skin surface. Extended dry times have been associated with such uncomfortable feel.

Thus, difficulty has been encountered in formulating an effective topical antimicrobial composition that also effectively moisturizes the user's skin intended for extended residence on the user's skin, as opposed to a soap or composition intended to be rinsed off the skin. Particular difficulty has been experienced in accomplishing this, and creating a formulation that still exhibits a relatively short dry time.

There exists a need in the field of topically applied broad spectrum antimicrobial compositions for compositions that achieve desired levels of antimicrobial efficacy while improving moisturization and feel, despite the necessary presence of certain harsh ingredients for antimicrobial effect. Even more preferred would be such a composition that exhibits a pleasant feel and avoids extended dry times.

### SUMMARY OF THE INVENTION

The invention provides an extended resident topical antimicrobial composition comprising an active antimicrobial and moisturizing ingredients that meets antimicrobial efficacy requirements and improves the condition of the user's skin as well. It has been surprisingly discovered that such a composition can be developed which not only exhibits broad spectrum antimicrobial efficacy levels at a desired standard, but which also exhibits an improved skin moisturization property without significant chemical interference with the active antimicrobial ingredient and which counteracts the harsh effects of the ingredients. It has further been discovered that certain combinations of moisturizing agents actually improve moisturization beyond the moisturization effects associated with the moisturizing ingredients individually. It has further been discovered that the desired efficacy and therapeutic effects can be accomplished with a formulation that still exhibits a relatively short dry time to avoid unpleasant feel. The invention is particularly useful to healthcare workers, whose usage of hand sanitizers can be on a daily and frequent basis.

The invention provides an extended resident topical antimicrobial composition comprising: a) an antimicrobial agent; and b) a moisturization component comprising a combination of a saccharide isomerate and carbohydrate complex together with glycerin. In a preferred embodiment, the antimicrobial agent is a broad spectrum antimicrobial agent such as alcohol.

The invention further provides a method of topically sanitizing and moisturizing skin comprising applying to said skin an extended resident topical antimicrobial composition comprising: a) an antimicrobial agent; and b) a moisturization component comprising a combination of a saccharide isomerate and carbohydrate complex together with glycerin.

One advantage of the invention is that it achieves the balance of antimicrobial effectiveness, while at the same time improving the condition of the user's skin over long term use, and minimizing dry time to avoid unpleasant feel on the user's hands associated with prolonged skin surface “wetness”.

### DETAILED DESCRIPTION OF THE INVENTION

As used herein, the term “resident” within the context of topical application is meant to refer to a formulation and context of use intended to remain on the user's skin for some period of time after its application, in the absence of subsequent rinsing, washing or other removal methods. The phrase “extended resident” is meant to refer to residency that occurs for relatively long periods of time, even indefinitely, once applied to the skin.

In general, the composition of the invention is an extended resident topical antimicrobial composition intended to remain on the skin for a relatively prolonged period of time while delivering antimicrobial effect to the skin surface. An important aspect of the invention is that despite the prolonged residence on the skin surface of an antimicrobial agent, the formulation also provides moisturizing effect to the skin which actually increases over time. The result is a “hand sanitizer” composition that at the same time is beneficial to the user's skin.

The extended resident topical antimicrobial composition comprises an antimicrobial agent and a moisturizing agent comprising a saccharide isomerate. The saccharide isomerate can be in the form of a complex formed by the combination of the saccharide isomerate and carbohydrates in aqueous solution. In one embodiment, the moisturizer comprises a saccharide isomerate of D-glucose in combination with an aqueous solution of carbohydrates. Preferably, the moisturizer further comprises glycerin in combination with the saccharide isomerate. The moisturizing agent can be present in an amount ranging from about 0.1% by weight to about 5% by weight of the composition, preferably from about 0.01% by weight to about 2%. In a preferred embodiment, saccharide isomerate is present in about 0.4% by weight and glycerin is present in about 0.4% by weight of the composition.

Aside from saccharide isomerate and glycerin, secondary moisturizers can be added to the composition of the invention as well. Suitable secondary moisturizers fur use in topical formulations include, but are not limited to, lanolin, pentylene glycol, and sodium pyrrolidone carboxylic acid.

A variety of antimicrobial agents can be used in accordance with the invention, preferably broad spectrum antimicrobial agents. Antimicrobial agents include, but are not limited to, chlorophenols, biguanides, iodophors, biologically active salts, and alcohols. Each of these antimicrobial classes exhibits differences in speed, persistence, and spectrum of activity. For example, chlorhexidine can be used to provide a formulation with increased persistence, and alcohol can be used to provide a formulation with “quick kill” properties.

In a preferred embodiment, alcohol is used as the antimicrobial agent. The alcohol can be ethanol, isopropanol, or mixtures thereof, such as a mixture of about 95% ethanol and about 5% isopropanol. The antimicrobial agent can be present in an amount ranging from about 40% by weight to about 90% by weight of the composition, preferably from about 50% to about 70%. In a preferred embodiment, alcohol (95% ethanol, 5% isopropanol) is present in about 59% by weight.

The composition of the invention can further comprise secondary ingredients provided that such secondary ingredients do not substantially interfere with the advantageous properties associated with the invention. Secondary ingredients that can be included in the composition are solvents, vitamins, additional moisturizers, surfactants, thickeners or conditioners, coloring agents, fragrances, and the like.

Solvents can be used to combine formulation ingredients in order to appropriately deliver the formulation to the user's skin. Solvents include, but are not limited to, alcohols, ethers, and ketones—each of which exhibit differences in solubility, odor, and drying potential. In a preferred embodiment, water is used as a solvent. Water as the solvent can be present in an amount ranging from about 10% by weight to about 55% by weight of the composition, preferably from about 30% to about 40%, most preferably in about 38% by weight of the composition.

The composition of the invention can further comprise vitamins as well. Vitamins can be used to impart numerous benefits, such as the promotion of healing and reduction of oxidation. Vitamins that can be used in topical formulations include vitamin A, C, D and E. While different vitamins and various combinations of vitamins can be used in accordance with the invention, preferred vitamins include vitamin E (DL-α-tocopherol) and vitamin B5 (d-panthenol). Vitamin E and vitamin B5 each can be present in an amount ranging from about 0.01% to about 5% by weight of the composition, preferably from about 0.01% to about 1%. In a preferred embodiment, vitamin E (DL-α-tocopherol is present in about 0.03% by weight and vitamin B5 (d-panthenol) is present in about 0.10% by weight.

Alpha hydroxy acids can be used in the composition of the invention as well. Alpha hydroxy acids are used in topical formulations to promote exfoliation and minimize skin flakiness. A variety of alpha hydroxy acids can be used in topical formulations, including glycolic acid, lactic acid, and hydroxycaprylic acid. A preferred alpha hydroxy acid for use in the invention is gluconolactone. Alpha hydroxy acids can be present in amount ranging from about 0.01% by weight to about 5% by weight of the composition, preferably from about 0.01% to about 0.5%. In a preferred embodiment, gluconolactone is present in an amount of about 0.05% by weight of the composition.

The composition of the invention can further comprise a surfactant. Surfactants can be used in topical formulations as wetting agents, to provide detergency and enhance foaminess. Suitable surfactants for topical formulations include, but are not limited to, glycerol monostearate, cocamidopropyl betaine, and lauramine oxide. Different surfactants and combinations of surfactants can be used in the invention. Preferred surfactants for use in the composition of the invention are nonionic surfactants. More preferred is stearamine oxide, which is both a nonionic surfactant and conditioning agent. Surfactants can be present in an amount ranging from about 0.01% by weight to about 5% by weight of the composition, preferably from about 0.01% to about 2%. In a preferred embodiment, stearamine oxide is present in an amount of about 1% by weight.

Thickeners and conditioners can be used in the composition to enhance viscosity and cosmetic attributes. Suitable topical thickeners or conditioners include carbomer, hydroxymethyl cellulose, and chitosan. Preferred for use in the invention as a thickener/conditioner is polyquaternium-10. The thickener/conditioner can be present in an amount ranging from about 0.1% by weight to about 3% by weight of the composition, preferably from about 0.5% to about 1.25% by weight. In a preferred embodiment, polyquaternium-10 is present in about 0.75% by weight of the composition.

Other additional ingredients that can be used include pigments, dyes, opacifiers, fragrances, and the like.

### EXAMPLE 1

### Preparation of the Resident Topical Anti-Microbial Composition

A composition in accordance with the invention was prepared as follows. In a stainless steel mixing vessel with both top and side-sweep agitators, ethanol and DL-α-tocopherol were initially mixed together under continuous agitation. After the DL-α-tocopherol was completely dissolved in solution, ambient water was added. Polyquaternium-10 was then added along with glycerin. The resulting solution was mixed until a clear smooth gel formed. Subsequently, saccharide isomerate and stearamine oxide were slowly added to the mixing vessel, and the resulting solution was mixed until the stearamine oxide was completely in solution. Finally, gluconolactone and d-panthenol were added and the solution was mixed until a clear homogenous solution was obtained.

The ethanol used to prepare the formula was SD alcohol, 200 proof (ATF 3C) (95% ethanol and about 5% isopropyl alcohol) (obtained from Equistar Chemicals, Houston, Tex.). Stearamine oxide used was Mackamine™ SO (obtained from McIntyre Group, University Park, Ill.). Polyquaternium-10 was Celquat™ SC-230M (obtained from National Starch, Bridgewater, N.J.). Saccharide isomerate/carbohydrate complex used in the formulation was Pentavitimm (available from Centerchem, Norwalk, Conn.).

The resulting composition contained the following Formula 1:

Formula 1 Anti-Microbial Composition of Invention Containing Combined

| | | Amount |
|---|---|---|
| Ingredient: | Function: | (wt %): |
| Deionized water | Solvent | 38.27 |
| Ethanol | Antimicrobial agent | 59.00 |
| DL-α-tocopherol | Vitamin E/skin | 0.03 |
| | conditioning agent | |
| Gluconolactone | Alpha hydroxy acid/skin | 0.05 |
| | conditioning agent | |
| d-panthenol | Vitamin B5/skin | 0.10 |
| | conditioning agent | |
| Stearamine oxide | Surfactant | 1.00 |
| Polyquaternium-10 | Thickener/conditioner | 0.75 |
| Glycerin | Moisturizer | 0.40 |
| Saccharide isomerate/ | Moisturizer | 0.40 |
| carbohydrate complex | | |
| Total: | | 100.00 |

### EXAMPLE 2

### Comparative In-Vitro Antimicrobial Efficacy Data

In a similar manner to the preparation of Formula 1, two additional topical antimicrobial formulas were prepared to compare different moisturizer components with the formulation of the invention. In all three formulas, ingredients remained identical and were present in identical weight % amounts, except for the presence and amounts of glycerin and saccharide isomerate as moisturizer components. In Formula 2, only glycerin was present as the moisturizer component. In Formula 3, only saccharide isomerate was present as the moisturizer component. In all three formulas, the total weight percent of moisturizer component present, irrespective of its ingredient(s), was 0.80. The formulas used in the experiment are set forth in the following table:

TABLE 1
Comparative Formulations

| Ingredient | Formula 1 | Formula 2 | Formula 3 |
|---|---|---|---|
| Deionized water | 38.27 | 38.27 | 38.27 |
| Alcohol | 59.00 | 59.00 | 59.00 |
| DL-α-tocopherol | 0.03 | 0.03 | 0.03 |
| Gluconolactone | 0.05 | 0.05 | 0.05 |
| d-panthenol | 0.10 | 0.10 | 0.10 |
| Stearamine oxide | 1.00 | 1.00 | 1.00 |
| Polyquaternium-10 | 0.75 | 0.75 | 0.75 |
| Glycerin | 0.40 | 0.80 | 0.00 |
| Saccharide isomerate/ | 0.40 | 0.00 | 0.80 |
| carbohydrate complex | | | |
| Total Weight % | 100.00 | 100.00 | 100.00 |

The antimicrobial efficacy for each of the formulas was evaluated in accordance with the In Vitro Time Kill Test procedures. Approximately 24 hours before initiating the study, the bacteria were propagated on soybean-casein digest agar (TSA=tryptic soy agar) plates and incubated at 35° C.±2° C. for bacteria, and at 25° C.±2° C. for 48 hours for yeast. Immediately prior to the testing procedure, well-isolated colonies were selected and transferred by sterile loop to 4-5 ml sterile physiological solution until a turbidity of 0.5 on McFarland Index Standard was reached. Accordingly, the suspensions contained approximately 1.0×10⁸ CFU/ml.

The inoculum titer was then prepared. An initial population was determined for each suspension by mixing 0.2 ml of the suspension with 1.8 ml sterile saline. A series of ten-fold dilutions (10⁻¹, 10⁻², 10⁻³, 10⁻⁴ and 10⁻⁵) was made by adding 0.22 ml into 2.0 ml sterile neutralizing solution containing lecithin, Tween 80 and sodium thiosulfate and vortex mixing. 0.2 ml aliquots of the dilutions were inoculated in duplicate onto an agar surface of TSA. The plates were then incubated at appropriate temperature and time parameters in an incubator: for bacteria at 35° C.±2° C. for 24 hours; and for yeasts 25° C. 2° C. for 48 hours.

For the testing procedure, 0.2 ml aliquot of each challenge suspension containing approximately 1.0×10⁸ CFU/ml was inoculated into a test tube containing 1.8 ml of test product (of Formulas 1, 2 and 3) and mixed thoroughly using a vortex mixer. The challenge microbial cells were exposed to each test product for 15 second and 1 minute exposure times.

After lapse of each exposure time, 0.22 ml was removed from the reaction tube into another test tube containing 2 ml sterile neutralizing solution. Ten-fold dilutions (10⁻¹, 10⁻² and 10⁻³) were made by placing 0.22 ml into 2 ml of sterile neutralizing solution.

Enumeration of microorganisms was done by standard plate count procedure. 1 ml aliquots of all 10⁻¹ dilutions were inoculated in duplicate, and 0.2 ml aliquots of the remaining dilutions were inoculated in duplicate onto the agar surface of TSA plates. The plates were then incubated for appropriate temperature and time parameters as described herein above for bacteria and yeasts. Following incubation, the growth colonies on the plates were then counted manually using hand tally counter. The raw CFU data was collected and converted to Log. Log reduction was calculated by subtracting the post treatment Log counts from the Log of the inoculum titer.

TABLE 2
Comparative Antimicrobial In-Vitro Efficacy Results

| Test Microorganism | Formula | Exposure Time | | Log Reduction |
|---|---|---|---|---|
| Ps. aeruginosa | Formula 1 | 15 | sec | >6.25 |
| ATCC# 15442 | | 1 | min | >6.25 |
| Gram (−) bacteria | | | | |
| | Formula 2 | 15 | sec | >6.25 |
| | | 1 | min | >6.25 |
| | Formula 3 | 15 | sec | >6.25 |
| | | 1 | min | >6.25 |
| E. coli | Formula 1 | 15 | sec | >6.12 |
| ATCC# 11229 | | 1 | min | >6.12 |
| Gram (−) bacteria | | | | |
| | Formula 2 | 15 | sec | >6.12 |
| | | 1 | min | >6.12 |
| | Formula 3 | 15 | sec | >6.12 |
| | | 1 | min | >6.12 |
| Ent. faecalis | Formula 1 | 15 | sec | >5.8 |
| ATCC# 29212 | | 1 | min | >5.8 |
| Gram (+) bacteria | | | | |
| | Formula 2 | 15 | sec | >5.8 |
| | | 1 | min | >5.8 |
| | Formula 3 | 15 | sec | >5.8 |
| | | 1 | min | >5.8 |
| St. aureus | Formula 1 | 15 | sec | >6.02 |
| ATCC# 6538 | | 1 | min | >6.02 |
| Gram (+) bacteria | | | | |
| | Formula 2 | 15 | sec | >6.02 |
| | | 1 | min | >6.02 |
| | Formula 3 | 15 | sec | >6.02 |
| | | 1 | min | >6.02 |
| Candida albicans | Formula 1 | 15 | sec | >5.09 |
| ATCC# 10231 | | 1 | min | >5.09 |
| Yeast | | | | |
| | Formula 2 | 15 | sec | >5.09 |
| | | 1 | min | >5.09 |
| | Formula 3 | 15 | sec | >5.09 |
| | | 1 | min | >5.09 |

As can be seen from the data, the formulation prepared according to the invention exhibits comparable antimicrobial efficacy as compared to Formulas 2 and 3. Therefore, it can also be determined from the results that the moisturizer component used in the inventive composition, i.e., the combination of saccharide isomerate and glycerin in the formulation prepared according to the invention does not adversely affect in vitro antimicrobial efficacy.

### EXAMPLE 3

### Comparative Moisturization Study

A study was performed to compare the skin moisturization potential of various topical antimicrobial formulations on subjects with dry skin. The moisturization effects of the topical antimicrobial composition of the invention (Formula 1) was evaluated and compared to that of Formula 2 (glycerin only) and Formula 3 (saccharide isomerate only). In addition, commercialized compositions Purell® available from Gojo Industries, Inc., Akron, Ohio), Endure® 320 (available from Ecolab, Inc., St. Paul, Minn.) and a control formulation (no moisturizing agents present) were included in the study. The Purell® formulation contains 62% ethanol and glycerin, among other ingredients. The Endure® 320 formulation contains 62% ethanol and glycerin, among other ingredients. The control formulation contained 59% weight of 200 proof SD alcohol (95% ethanol, 5% isopropyl alcohol) and 41% weight of deionized water.

Twelve female subjects ranging from 31 to 59 years of age were selected following a six-day wash-out period, during which only non-moisturizing cleansing bars (Neutrogena™) were used on the lower legs of the subjects in the absence of any moisturization products. Further, the subjects were to refrain from shaving their legs within 72 hours of the test date. Subjects were selected based on pre-treatment phase NOVA reading of less than 110 on their lower legs. NOVA measurements were taken using NOVA DPM 9003® (NOVA Technology Corporation, Portsmouth, N.H.).

The subjects were equilibrated in a controlled environment of less than 50% relative humidity and 70°±3 F. for 30 minutes before undergoing any measurements. At baseline, each of the subjects had three test sites demarcated (using a skin marker) on the outer aspect of one lower leg measuring 4 cm×4 cm and four test sites demarcated on the outer aspect of the contra-lateral leg measuring 4 cm×4 cm. Six of the sites were treated with the test formulations, and the seventh site served as a control (untreated) site. Baseline measurements were taken using the NOVA meter of each test site. The site treatments were rotated from right to left and top to bottom among the subjects to remove positional bias.

Next, approximately 20 μl of each test formulation was applied to each of the respective test sites using a finger cot and spread evenly over the site. NOVA meter measurements taken immediately upon drying (at time 0). NOVA readings were taken at each test site in duplicate after 30 seconds following application. The test formulations were re-applied at approximately 1 hour later, followed by additional NOVA meter readings. The procedure was repeated at hours 2, 3 and 4 for a total of 5 applications at each test site of the designated test formulation. The subjects were required to remain in the test facility throughout the duration of the teasing without drinking excessive water, smoking or eating.

The NOVA data for each subject was compiled and statistical analysis was performed using ANOVA followed by Tukey-Kramer Multiple Comparisons Test utilizing the deltas from baseline to one hour, baseline to two hours, three and four hours respectively.

The group mean NOVA readings for each test formulation and the untreated control at each evaluation time are set forth in the following table:

TABLE 3
NOVA Group Means

| Treatment | Baseline | 0 hour | 1 hour | 2 hour | 3 hour | 4 hour |
|---|---|---|---|---|---|---|
| Formula 1 | 95.67 | 102.42 | 107.17 | 118.50 | 114.92 | 126.17 |
| Formula 2 | 95.75 | 102.17 | 108.17 | 110.83 | 119.17 | 126.00 |
| Formula 3 | 95.92 | 98.17 | 110.25 | 112.08 | 108.17 | 119.92 |
| Purell ® | 96.33 | 96.08 | 102.08 | 100.00 | 96.75 | 98.42 |
| Formula | | | | | | |
| Endure ® | 97.25 | 101.33 | 107.67 | 108.67 | 104.50 | 106.75 |
| 320 Formula | | | | | | |
| Ethanol | 96.00 | 95.67 | 94.83 | 97.08 | 97.50 | 98.25 |
| Control | | | | | | |
| Untreated | 97.42 | 96.67 | 96.08 | 96.25 | 96.67 | 96.17 |
| control | | | | | | |

As can be seen from the above data, group mean scores for Formulas 1, 2 and 3 were comparatively higher than the Purell® and Endure® 320 formulations. Furthermore, Formulas 1, 2 and 3 exhibited an additive moisturization effect over time, whereas Purell® and Endure® exhibited decreasing moisture readings at two and three hour evaluation times, respectively. At the two hour and four hour evaluation times, Formula 1 of the invention exhibited the highest moisturization effects of the test formulations, even within the test formulation subgroup of Formulas 1, 2 and 3.

The NOVA data was calculated in terms of percent change from baseline as well. The percent change data for each of the test formulations is set forth in the following table.

TABLE 4
Percent Change in NOVA Readings

| | Baseline | Baseline | Baseline | Baseline | Baseline |
|---|---|---|---|---|---|
| | to 0 | to 1 | to 2 | to 3 | to 4 |
| | hour % | hour % | hour % | hour % | hour % |
| Treatment | change | change | change | change | change |
| Formula 1 | 7.1% | 12.0% | 23.9% | 20.1% | 31.9% |
| Formula 2 | 6.7% | 13.0% | 15.7% | 24.5% | 31.6% |
| Formula 3 | 2.3% | 14.9% | 16.8% | 12.8% | 25.0% |
| Purell | −0.3% | 6.0% | 3.8% | 0.4% | 2.2% |
| Endure 320 | 4.2% | 10.7% | 11.7% | 7.5% | 9.8% |
| Ethanol | −0.3% | −1.2% | 1.1% | 1.6% | 2.3% |
| control | | | | | |
| Untreated | −0.8% | −1.4% | −1.2% | −0.8% | −1.3% |
| control | | | | | |

As can be seen from the percent change data, Formula 1 of the invention produced the highest increase in skin moisturization over the entire study with a final group percent change in moisturization over baseline of 31.9%. Formula 2 produced a lower overall percent change of 31.6%, and Formula 3 produced a 25% increase in skin moisturization over baseline. Commercial formulas Purell® and Endure® produced percent changes over baseline of 2.2% and 9.8% respectively. Accordingly, Formula 1 of the invention produced the most significant long term skin moisturization effect as compared to the other formulations in the study.

### INDUSTRIAL APPLICABILITY

The extended resident topical antimicrobial composition of the invention is useful as a hand sanitizer when washing with soap and water is inconvenient. The composition of the invention is formulated to reduce adverse effects on the skin from frequent use, and even improve the condition of the user's skin. The invention is particularly useful to healthcare workers to reduce and avoid healthcare-acquired infection while accommodating daily and frequent use of the composition.

The invention has been described herein above with reference to various and specific embodiments and techniques. In will be understood by one of ordinary skill in the art, however, that reasonable variations and modifications to such embodiments and techniques can be made without significantly departing from either the spirit or scope of the invention as defined by the following claims.

What is claimed is:

1. An extended resident topical antimicrobial composition comprising:
an antimicrobial agent; and
a moisturizer comprising a saccharide isomerate.

2. The composition according to claim 1, wherein said saccharide isomerate is a complex formed by isomerization of D-glucose in combination with an aqueous solution of carbohydrates.

3. The composition according to claim 1, wherein said antimicrobial agent is a broad spectrum antimicrobial agent.

4. The composition according to claim 1, wherein said antimicrobial agent comprises ethanol.

5. The composition according to claim 1, wherein said moisturizing agent further comprises glycerin.

6. An extended resident topical antimicrobial composition comprising:
a) alcohol;
b) a saccharide isomerate and carbohydrate complex; and
c) glycerin;
wherein said composition moisturizes skin.

7. The composition according to claim 6, wherein said saccharide isomerate and carbohydrate complex, and said glycerin are present in percent weight amounts in a ratio of about 1 to 1.

8. The composition according to claim 6, wherein ethanol is present in an amount of about 59% by weight.

9. The composition according to claim 6, comprising:
a) water;
b) alcohol;
c) α-tocopherol;
d) gluconolactone;
e) d-panthenol;
f) stearamine oxide;
g) polyquaternium-10
h) glycerin; and
i) saccharide isomerate/carbohydrate complex.

10. The composition according to claim 9, comprising:
a) water present in an amount of about 38.27% by weight;
b) alcohol present in an amount of about 59% by weight;
c) α-tocopherol present in an amount of about 0.03% by weight;
d) gluconolactone present in an amount of about 0.05% by weight;
e) d-panthenol present in an amount of about 0.10% by weight;
f) stearamine oxide present in an amount of about 1.00% by weight;
g) polyquaternium-10 present in an amount of about 0.75% by weight;
h) glycerin present in an amount of about 0.40% by weight; and
i) saccharide isomerate/carbohydrate complex present in an amount of about 0.40% by weight.

11. A method of topically sanitizing and moisturizing skin comprising applying to said skin an extended resident topical antimicrobial composition, said composition comprising:
an antimicrobial agent; and
a moisturizer comprising a saccharide isomerate.

12. The method according to claim 11, wherein said saccharide isomerate is a complex formed by isomerization of D-glucose in combination with an aqueous solution of carbohydrates.

13. The method according to claim 11, wherein said antimicrobial agent is a broad spectrum antimicrobial agent.

14. The method according to claim 12, wherein said antimicrobial agent comprises ethanol.

15. The method according to claim 11, wherein said moisturizing agent further comprises glycerin.

16. The method according to claim 11, wherein the topical antimicrobial composition comprises:
a) alcohol;
b) a saccharide isomerate and carbohydrate complex; and
c) glycerin.

17. The method according to claim 16, wherein said saccharide isomerate and carbohydrate complex, and said glycerin, are present in a weight percent ratio of about 1 to 1.
